(19) United States
(12) Patent Application Publication
(10) Pub. No.: US 2014/0343691 A1
(43) Pub. Date: Nov. 20, 2014

(54) Systems and Methods for a Wireless Myoelectric Implant

(71) Applicant: Ripple LLC, Salt Lake City, UT (US)

(72) Inventors: Kenneth Shane Guillory, Salt Lake City, UT (US); Scott Darold Hiatt, Sandy, UT (US); Ronald E. Madsen, JR., Salt Lake City, UT (US); Daniel McDonnall, Salt Lake City, UT (US); Christopher Farand Smith, North Salt Lake, UT (US); Daniel R. Merrill, Salt Lake City, UT (US); Robert E. Askin, III, Salt Lake City, UT (US)

(73) Assignee: Ripple LLC, Salt Lake City, UT (US)

(21) Appl. No.: 14/281,739

(22) Filed: May 19, 2014

Related U.S. Application Data
(60) Provisional application No. 61/824,742, filed on May 17, 2013.

(51) Int. Cl. A61F 2/72

(52) U.S. Cl. CPC A61F 2/72; A61F 2002/482
USPC 623/25

(57) ABSTRACT

This disclosure relates to a systems and methods for control of external devices, such as a prosthesis, using biopotential signals from electrodes in communication with existing muscles or nerves of a patient. More specifically, but not exclusively, this disclosure relates to systems that include wireless transmission of biopotential signals and a multichannel bioamplifier configured to receive biopoential signals. The system may also be configured to stimulate excitable tissue within the body.

### RELATED APPLICATIONS

This application claims the benefit of U.S. Provisional Patent Application 61/824,742, filed May 17, 2013 and titled “SYSTEMS AND METHODS FOR A WIRELESS MYOELECTRIC IMPLANT”.

### TECHNICAL FIELD

This disclosure relates to a systems and methods for control of external devices, such as a prosthesis, using biopotential signals from electrodes in communication with existing muscles or nerves of a patient.

### BRIEF DESCRIPTION OF THE DRAWINGS

Non-limiting and non-exhaustive embodiments of the disclosure are described, including various embodiments of the disclosure with reference to the figures, in which:

FIG. 1 illustrates a functional block diagram of a system for controlling external devices, such as a prosthetic device, using a wireless myoelectric implant consistent with embodiments disclosed herein.

FIG. 2 illustrates a method for controlling external devices, such as a prosthetic device, using a wireless myoelectric implant consistent with embodiments disclosed herein.

A detailed description of systems and methods consistent with embodiments of the present disclosure is provided below. While several embodiments are described, it should be understood that the disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

### DETAILED DESCRIPTION

Prosthetic devices may be designed with different goals in mind. These goals may depend on the type and location of the amputation and the needs of the patient. For example, some prosthetic devices may be designed with appearance in mind rather than controllability. Advances in plastics and pigments matched to the patient's skin tone may allow a prosthetic device to take on a life-like appearance. Other prosthetic devices may be designed with usability and function as a central purpose. Functional prosthetic devices can be controlled in a variety of ways. Body powered prosthetic devices can be controlled by cables connecting them to elsewhere in the body. Powered prosthetics can have motors and may be controlled by the patient using buttons or switches.

A prosthetic device may also be controlled using existing muscle groups in the residual limb that the user may be able to voluntarily activate. By connecting sensors to these muscles the patient may be able to control the prosthetic device by activating the remaining muscles. The sensors may be connected to a processor and amplification circuitry to create movement in a prosthetic. As used in the present disclosure, the term myoelectric prosthesis refers to devices that use biopotential signals or potentials from voluntarily activated muscles to control the movements of a prosthesis.

In connection with a myoelectric prosthesis, biopotential signals may be collected via an electrode, lead, or sensor. Leads are structures that contain one or more electrodes or sensors that are individually placed, or placed in conjunction with other leads. Biopotential channels are electrical differences recorded between one or more electrodes. Electrodes/leads/sensors may be placed on the surface of the skin or can be implanted. A biopotential signal receiving device may also be implanted and may connect with an external transceiver via a wireless communication channel. This device may also provide stimulation feedback to the muscle or nerve to simulate temperature, pressure, or movement sensation to the patient.

According to various embodiments, systems and methods consistent with the present disclosure may include a wireless multichannel myoelectric implant. In some embodiments a wireless multichannel implant may be used to acquire biopotential signals from implanted electrodes. Representations of the acquired biopotential signals may be transmitted wirelessly to a system configured to receive, processes, and utilize the signals to control a myoelectric prosthesis.

The embodiments of the disclosure will be best understood by reference to the drawings, wherein like parts may be designated by like numerals. The components of the disclosed embodiments, as generally described and illustrated in the figures herein, could be arranged and designed in a wide variety of different configurations. Thus, the following detailed description of the embodiments of the systems and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified.

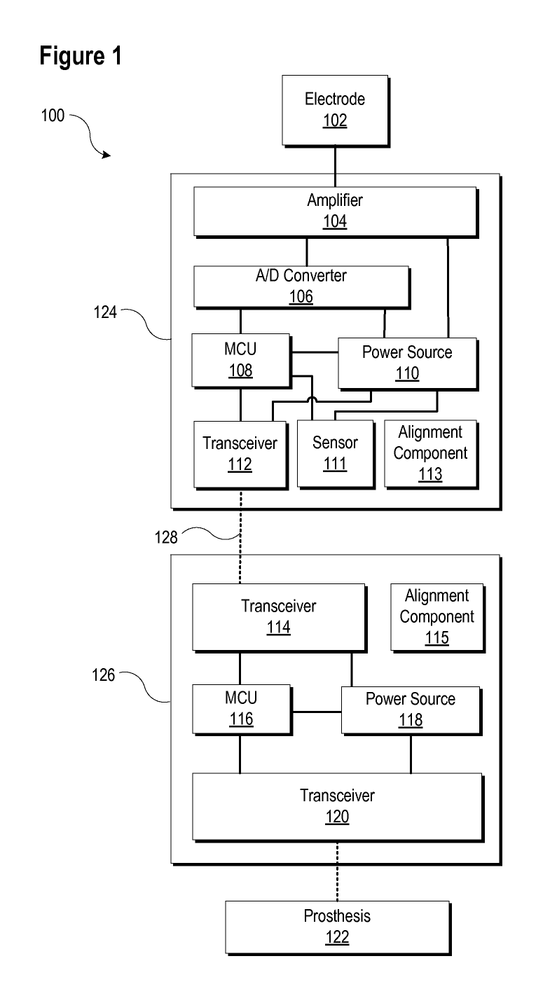

FIG. 1 illustrates a functional block diagram of a system 100 including an electrode lead 102, an implant 124, an external component 126, and a prosthesis 122 consistent with certain embodiments disclosed herein. According to various embodiments, one or more of electrode lead 102 and/or implant 124 may be implanted. These devices may have features designed to hold implanted structures in place, including but not limited to: screw points, suture holes, anchor points, and special films. Certain features may be reinforced by supplemental materials such as metal rings or polymer fibers to prevent tearing. The device may have terminal fixation points that may penetrate intramuscularly and be safely left in the body after explantation.

An implantable manifestation of implant 124, for example, may have electronics hermetically sealed in a small implantable enclosure. According to various embodiments, implant 124 may comprise an amplifier 104, which may be capable of multiple channels of bioamplification. Amplifier 104 may exhibit a relatively fast settle time to permit concurrent stimulation and recording with electrodes in close proximity

Implant 124 may further comprise an A/D converter 106 that is configured to convert the biopotential signals received from amplifier 104 to digital signals.

A microcontroller unit (MCU) 108 may perform signal processing operations and/or implement other functions. MCU 108 may comprise a microcontroller, microprocessor, programmable logic device, or any system used to perform signal processing and perform other functions described herein. According to some embodiments, MCU 108 may be omitted from implant 124, and signal processing capabilities may instead be performed by external component 126. As illustrated in FIG. 1, external component 126 may also comprise an MCU 116. Still further, MCU 108 and MCU 116 may, according to some embodiments, be implemented using a computer, a PDA, a tablet, a phone, or a remote control.

According to some embodiments, system 100 may communicate wirelessly to a computer to interact with a training system. The training system may include an external transceiver independent from a prosthetic device and software designed to allow the patient to visualize electromyography (EMG) activity. The device may have a training system designed especially for infants, toddlers, and/or other young children with congenital limb deficiency to learn to use EMG to control a prosthetic. The device may have a training system that uses cameras and/or mirrors that give the illusion of possessing the missing limb.

According to some embodiments a sensor 111 may be included in implant 124 in order to provide additional control for prosthesis 122. According to one embodiment, sensor 111 may comprise an accelerometer capable of detecting mechanical perturbation that can be used to control the prosthesis 122. According to various embodiments, other types of sensors may also be utilized, such as temperature sensors, pressure sensors, moisture sensors, etc.

According to some embodiments, an alignment component 113 may also be included in order to facilitate the alignment of transceiver 112 and transceiver 114. In various embodiments, transceiver 112 and transceiver 114 may each comprise a coil that may be utilized for data transmission. Alignment component 113 may operate in conjunction with alignment component 115 in external component 126. According to some embodiments, alignment component 113 and alignment component 115 may comprise magnets. Further, according to certain embodiments, alignment component 113 and alignment component 115 may be disposed internally or externally to implant 124 and external component 126, respectively. In still other embodiments, alignment component 113 and alignment component 115 may interact with a tool for intraoperatively aligning the position of the transceiver 112 and transceiver 114.

Implant 124 may comprise an enclosure made of ceramic, metal, epoxy, polymeric material or any combination thereof. Hermetic enclosures provide gas-tight areas that are created by metal, glass and ceramic enclosures. Implant 124 may include a hermetic enclosure to encapsulate portions of the implant components. Additional surgical materials may be implanted, such as films, screws, etc., to improve the tolerance or biocompatibility or fixture of the device and/or health of skin or other tissues over or close to implant 124. The device may include features such as tapers or edges to facilitate easier tunneling through tissue during surgical placement. Implant 124 may include non-stick or non-adhesive coatings on surfaces to make explantation of the wires and/or enclosure easier and the device may have exposed metal areas that can serve as electrodes for recording, stimulating, grounding and/or referencing relative to other electrodes. The enclosure enclosing implant 124 may include electrical feedthroughs to allow electrical and electronic coupling to the electrodes. These connections may be sealed with silicone or other material.

In certain embodiments electrode leads 102 may be configured to extract biopotential signals from extramuscular and/or intramuscular sites. Electrode leads 102 may, for example, be placed in the chest and/or shoulders, arms, hands, pelvic muscle, legs (upper and lower), or any other extramuscular or intramuscular site that may be used along with muscle decoding algorithms for control of prosthetic devices, computers, wheelchairs, robotic exoskeleton, and/or any other internal or external device. In certain embodiments electrode lead 102 may be comprised of multiple helically coiled wires housed in silicone tubing terminating in multiple extramuscular bands of electrodes. Electrode lead 102 may further include barbs, metal rings, or reinforced polymer for attachment to tissue and may be used to tether extramuscular electrodes.

According to some embodiments, electrode lead 102 may comprise electrodes configured to acquire biopotential signals as well as stimulation electrodes. Stimulation electrodes may be constructed of thin film materials shielded on one side to permit selective activation of re-innervated cutaneous sensory fibers without activating underlying muscle tissue. Stimulation and/or acquisition electrodes may be composed of polymers doped with conductive particles and may be composed of layers of insulating and conductive layers to create a thin flexible structure that uses conductive traces instead of wires for electrical connections. The electrode lead bundle may have marks that denote which side of the structure contains electrodes and the device may be designed so that the distal electrode array is no wider than the lead bundle to facilitate safe explantation of the device. In certain embodiments the lead and electrode array may have a thickness less than 500 μm. The surface area of a stimulation electrode, according to some embodiments, may be at least 200,000 μm². The electrodes and leads may be trimmable and/or have parts or covers used for tunneling or placement that can be broken off or trimmed after placement. The electrodes may consist of separate active electrodes bonded to cable wires, or the wires themselves may be exposed to form stimulating electrode sites. Electrode arrays may also be formed by de-insulating wires at multiple sites so that a single conductor forms multiple contact electrodes. The electrodes may be an exposed section of a continuous conductive polymer trace that extends back to the electronics enclosure. The electrode wires may be bonded together in places for easier electrode cable management and placement.

In certain embodiments electrode lead 102 may comprise electrodes configured to acquire biopotential signals and stimulation electrodes of different types. For example different types of electrodes may include: microwires, helically coiled wires, stainless steel wires, platinum electrodes, iridium electrodes, nerve cuffs around nerve roots, silicon-based multichannel penetrating nerve electrodes, intrafascicular electrodes, conductive polymer electrodes, and polymeric thin film electrodes. Electrode lead 102 may attach to a single hermetically sealed enclosure and also may have lead(s) that branch to multiple separate leads that terminate with electrodes and lead fixation components. The electrode lead 102 may include insulated and/or exposed shields or shield wires wrapped around the assembly for electrical shielding and EMC improvement. The electrode and cable assemblies may include ferrite chokes for EMC and/or MRI compatibility. The electrodes may include coils or other stress relief features for minimizing risk of electrode breakage and connections to the electrodes may be serviceable so that the enclosure or electrodes can be individually replaced. Electrodes in lead 102 may, according to some embodiments, have some surface treatment (e.g., carbon nanotube coating) to improve the recorded signal. Electrode leads 102 may comprise leads organized in packaging with a clip that fastens leads in place until use and electrodes may be placed using ultrasound to guide lead placement.

According to some embodiments, electrode lead 102 may include a feature at the distal end to allow the electrode lead 102 to be implanted by clamping to the feature and pulling it through to its intended location. In one manifestation of the device this feature may comprise a tab of nonconductive material. The device may be implanted using a specific tool developed to protect the electrode lead 102 as it is pulled through to its implantation site. This tool would allow the clinician to grasp the electrode lead 102 without causing damage. Electrode lead 102 may be placed by any surgical method, including endoscopic techniques. For example, the device may be placed using an intraluminal guidewire placement tool. This system may have a lead that can be passed out of the sterile field. In certain embodiments the implant device may require a minimally invasive insertion procedure that does not require general anesthesia. Implant device function may be confirmed using an intraoperative tool to inductively power the device. The implanted hermetic enclosure may be implanted subcutaneously in an area beneath the socket for a prosthetic device.

A power source 110 may be located internally or externally to implant 124. Power source 110 may be embodied as a rechargeable battery, an inductive device (i.e., an inductive coil for receiving power and/or data transmission), or any other suitable system used for providing power to implant 124. Power may be inductively drawn from the external transceiver though the wireless interface 128. According to some embodiments, a power source 110 may comprise a mechanical recharging system that generates electrical power using patient movement. According to still other embodiments, the implant 124 may be inductively powered without a battery by an external device similar to a cochlear implant. Still further, the implant 124 may include a medical grade single use battery that can be replaced. Implant 124 may be configured to transmit out battery configuration information, such as number of charge cycles, charge level, expected lifetime, etc. Implant 124 may further include protective circuitry for limiting overcharging of the batteries.

Transceivers 112 and 114 may communicate using a variety of technologies. For example, transceivers 112 and 114 may transmit signals by infrared transmission, reflected impedance transmission, and/or any applicable data transmission system. According to some embodiments, transmitted or received data may be recorded. Transceivers 112 and 114 may communicate wirelessly across a wireless communication channel 128.

External component 126 may be in communication with prosthesis 122. Signals received from electrode lead 102 may be transmitted to prosthesis 122 to induce a desired action or movement. Moreover, prosthesis 122 may generate and transmit information using system 100 that results in stimulation using electrode lead 102.

Although not specifically illustrated, system 100 may further include various components or devices for safety and other functions. For example, system 100 may include an RFID tag and/or radio opaque marking for identification of the device, an emergency shutoff device, an electrostatic discharge protection system, and/or a manual trigger for system verification.

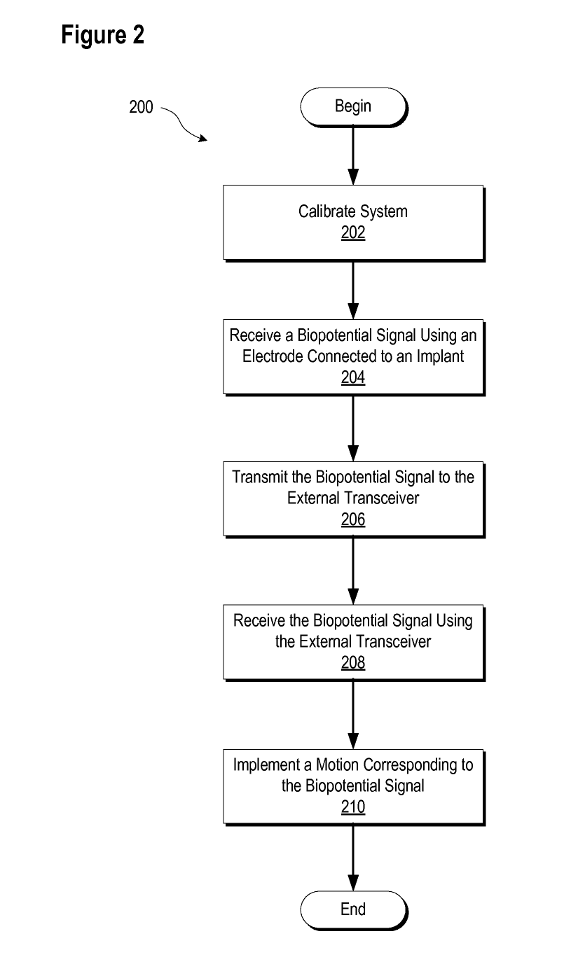

FIG. 2 illustrates an exemplary flow chart of a system for controlling a myoelectric prosthetic device using a wireless implant system consistent with embodiments disclosed herein. The illustrated method may be performed using, at least in part, a wireless myoelectric implant, electrodes or leads, a motorized prosthetic, an external transceiver, and/or any other suitable system or device.

At 202, a system may be calibrated. System calibration may include adjusting the strength of the magnetic coupling force between the internal and external magnets. System may be controlled by use of a magnet moved by the patient by proximity or gestures. The system may communicate with an external analog interface for confirming system function, system calibration, and/or patient training.

At 204 an electrode (or electrodes) may receive a biopotential signal from a contracting muscle. The electrode may be connected to an implant. According to various embodiments, the signal may digitized and/or processed by the by the implant. At 206 the signal may be transmitted to an external transceiver. At 208 the signal may be received by the external transceiver. At 210 a motion corresponding to the biopotential signal may be implemented by a prosthesis.

During the method of receiving and transmitting the signal for controlling the prosthetic, the signal may be processed by a microchip, processor, or any other device implemented to determine the desired characteristics. The signal may be used to determine speed, strength, and/or direction of the action requested of the prosthetic. The signal processing may occur in the implant, the external transceiver, the myoelectric prosthetic, and/or external computer/tablet/phone or any combination thereof. The signal data may be encrypted to prevent eavesdropping of critical information. The device may use error detection and/or error correction codes such as CRC to validate exchanged data before using it to configure the device. Signals may be used to acknowledge receipt of commands or changes of state and may be used to authenticate devices used for configuration. The device may be able to transmit raw EMG data out for visualization and configuration and may include data logging. The device may also have diagnostic modes to verify it is functioning before and after implantation. The diagnostic system may include means of measuring impedances to diagnose problems with the device. If diagnostic problems are detected a fail-safe shutdown mode may be implemented.

The device may have the capability to communicate with other implants in a patient and may allow for other implants to communicate with multiple external transceivers without interference. The device may be capable of down selecting the number of recording channels or go into a low power mode to decrease power consumption. The device may also have the capability to increase the sampling frequency for the remaining channels or adjust the gain for individual amplifier channels. The device may also go into deactivation mode for MRI scanning.

The implant may also transmit other critical information such as implant optimal alignment, error codes, status, diagnostics, or other information useful to determine health of the implant or patient. The device may also be used to extract signals from residual muscles from patients following targeted muscle innervation. The device may use physiological and/or mechanical signals to control stimulation, such as a pattern of gestures. Physiological and mechanical signals may also be used in combination to control the device. Stimulation via electrodes and leads may be used to activate re-innervated cutaneous sensory fibers to restore pressure, slip, and/or temperature perception.

The device may also be used to control authentication of implanted or external devices used to dispense personal identification, monetary, and other confidential information.

After the signal is processed for the desired movement the signal is delivered to the prosthesis 122. The device may allow for different modes of operation in which EMG control is used for different applications. For example a tool mode in which a greater degree of control is given to moderating a single repetitive task or a keyboard mode in which EMG signals normally used for wrist control are devoted to finger motion during typing.

The external module may be placed in a prosthetic device and may have a communication and/or powering coil that is attached via a cable to the prosthetic device for placement over the implant a short distance away from the prosthesis.

1. A system comprising:
an electrode configured to receive a biopotential signal;
an implant in communication with the electrode, the implant configured to generate a representation of the biopotential signal and to transmit the representation;
an external component in communication with the implant and configured to receive the representation;
a prosthesis in communication with the external component and configured to control the prosthesis based upon the representation.
